SEQUENCE LISTING (2 sequences)

SEQ ID NO: 1 - DNA, 10 bp, Artificial Sequence
  Description of Artificial Sequence Synthetic oligonucleotide
ggatcnnnnn

SEQ ID NO: 2 - DNA, 10 bp, Artificial Sequence
  Description of Artificial Sequence Synthetic oligonucleotide
gagtcnnnnn

(19) United States
(12) Patent Application Publication
(10) Pub. No.: US 2014/0087390 A1
(43) Pub. Date: Mar. 27, 2014

(54) METHOD AND SYSTEM FOR ANALYSIS OF PROTEIN AND OTHER MODIFICATIONS ON DNA AND RNA

(71) Applicant: NABsys, Inc., Providence, RI (US)

(72) Inventors: John S. Oliver, Bristol, RI (US); John Thompson, Warwick, RI (US)

(73) Assignee: NABsys, Inc., Providence, RI (US)

(21) Appl. No.: 14/036,509

(22) Filed: Sep. 25, 2013

Related U.S. Application Data
(60) Provisional application No. 61/705,983, filed on Sep. 26, 2012.
(60) Provisional application No. 61/774,216, filed on Mar. 7, 2013.

(51) Int. Cl. G01N 33/53; G01N 27/447

(52) U.S. Cl. CPC G01N 33/5308; G01N 27/447
USPC 435/6.19; 204/451

(57) ABSTRACT

A protocol and system for determining sites at which proteins directly bind to DNA or RNA, modify other proteins including histones, or bind to other proteins as well as determining sites at which DNA or RNA is modified is described herein. A simplified, highly accurate method for studying protein interactions with DNA or RNA and sites of DNA or RNA modification using nanodetector systems is provided.

### RELATED APPLICATIONS

This application claims the benefit of priority under 35 U.S.C. §119(e) to U.S. Provisional Patent Application Ser. No. 61/705,983 filed Sep. 26, 2012 and to U.S. Provisional Patent Application 61/774,216 filed Mar. 7, 2013; the entirety of both of these applications is incorporated herein by reference.

### FIELD OF INVENTION

Embodiments of the present invention relate generally to methods and apparatus for determining one or more regions on a DNA or RNA sample to which proteins of interest bind and/or one or more regions on a DNA or RNA sample that are modified by methylation or by another chemical moiety.

### BACKGROUND

Gene expression, differentiation, and development are modulated and controlled by DNA modifications and a variety of proteins acting either directly by binding DNA, by modifying other proteins including histones, or by binding other proteins. Understanding how these interactions occur and how they regulate key biological processes can provide a basis for improving medical and other outcomes. Such DNA modifications and protein interactions were initially characterized individually using chemical and enzymatic footprinting techniques. As microarray technology advanced, DNA modifications and protein interactions were studied in a highly parallel fashion so that many more genomic regions could be studied simultaneously.

One method for obtaining information about DNA modifications and protein binding to DNA is chromatin immunoprecipitation (referred to herein as “ChIP”). ChIP is employed to determine whether DNA modifications and specific proteins are associated with specific genomic regions, such as transcription factors on promoters or other DNA binding sites. ChIP is also used to determine specific locations in the genome associated with various histone modifications, thereby indicating the target of the histone modifiers.

Briefly, the ChIP method is as follows: protein and associated chromatin in a cell lysate are temporarily bonded or crosslinked to form DNA (chromatin)/protein complexes. The complexes are then sheared, and DNA fragments associated with the proteins of interest are selectively immunoprecipitated. The associated DNA fragments are then purified with the resulting DNA fragments presumed to be associated with the protein of interest in vivo. A method employing ChIP is described, for example, in U.S. Pat. No. 6,410,243 to Wyrick, et al., incorporated herein by reference in its entirety. The methodology for detecting DNA modifications is somewhat different. Because the modifications are already part of the DNA, no bonding or crosslinking is necessary. Instead, DNA is prepared from the source of interest, bound to proteins or other molecules with specificity for the desired modification, and then separated from the bulk DNA based on that binding. Typically, DNA is sheared prior to separation of the modified and unmodified DNA.

Next generation sequencing developments lead to further advancements with the introduction of ChIP Sequencing, (referred to herein as “ChIP-Seq”). ChIP-Seq provides broader coverage of the genome, allowing an even greater understanding of protein binding and histone modification. Similarly, sequencing of DNA enriched in modifications has been carried out and is sometimes referred to by different names such as methylated DNA precipitation (MeDIP) (Salpea et al., Nucleic Acids Res., 2012 August; 40(14):6477-94) or GLIB (glucosylation, periodate oxidation, biotinylation) or CMS (conversion of 5hmC to cytosine 5-methylenesulphonate) (Pastor et al., Nature, 2011 May 19; 473(7347):394-7); both references are incorporated herein in their entireties. These and related technologies will be referred to herein as “Mod-Seq”. There are additional methodologies for detecting modifications that employ treatments of DNA with methylation-specific enzymes or chemicals that chemically alter a base such that it is recognized differently by sequencing reactions (e.g. bisulfite treatments). Widespread characterization of such protein binding and protein modifications and DNA modifications have been collected by the ENCODE project across a variety of cell lines and conditions.

The overall workflow associated with ChIP-Seq and Mod-Seq is labor intensive. Once a chromatin target is isolated and fragmented, the ChIP process (described above) is carried out. The resulting DNA fragments are subjected to reverse crosslinking to remove protein (for ChIP-Seq), and then single-stranded DNA (ssDNA) extensions from the double-stranded DNA (dsDNA) fragments are repaired and modified (for both ChIP-Seq and Mod-Seq). Adapters are ligated to the dsDNA fragments, which are then isolated on a gel to separate the fragments by size. Selected fragments are then amplified using the polymerase chain reaction (PCR). Because most sequencing systems are very expensive to run, it is important not to sequence samples that are not optimal. Thus, fragments resulting from PCR are generally analyzed via gel electrophoresis or other sizing methods to ensure that the proper size fragments have been generated and a substantial fraction have both desired linkers ligated. The sample is then generally quantitated using real time PCR so the proper amount of sample can be used for sequencing. Finally, next generation sequencing may be employed to determine the sequence of each selected fragment, and thereby a target binding or modification site for the proteins of interest.

While ChIP-Seq and Mod-Seq data has been valuable, the technology has limitations. First, the methodology is tedious with many steps requiring a high level of expertise. The lengthy protocols may lead to difficulties in generating sufficient material for many proteins and modifications. Second, short reads used in ChIP-Seq and Mod-Seq may lead to difficulties in assessing long range interactions among proteins and modifications and whether proteins are binding to the same or different DNA molecules. Additionally, distinguishing multiple proteins or modifications using ChIP-Seq and Mod-Seq is difficult or impossible, thereby limiting the observation of more complex interactions. Thus, while ChIP-Seq and Mod-Seq have greatly furthered the understanding of many aspects of biological regulation, the technology remains limited in some respects. Therefore, an improved protocol for detecting protein binding and DNA modification on a genome-wide basis would provide valuable benefits.

### SUMMARY

For the sake of simplicity, although the description herein will primarily refer to protein binding to DNA or a DNA/protein complex and modifications to DNA, it is to be understood that, unless otherwise specified, these terms are intended to refer to protein binding to RNA and RNA/protein complexes and modifications to RNA as well.

In one aspect, embodiments of the present invention relate to a method for determining sites of protein binding to DNA or RNA or to sites that are modified in DNA or RNA. Broadly, embodiments of the invention include the steps of providing a sample of a DNA/protein complex, an RNA/protein complex, modified DNA or modified RNA to be analyzed, translocating the sample through a nanodetector having a detection zone, detecting and monitoring an electrical property in the detection zone, and analyzing the electrical property to determine at least one site of the sample to which a protein is bound or at which a modification is present. Changes in the electrical property allow discrimination of i) the absence of the sample in the detection zone, ii) the presence of a portion of the sample lacking a bound protein or modification in the detection zone, and iii) the presence of a portion of the sample including a bound protein or modification in the detection zone.

The sample may be isolated from a biological sample or it may be created in vitro, and the protein may be crosslinked or otherwise bound to the DNA or RNA. Modifications to DNA or RNA may include methylation, hydroxylation or glucosylation.

Detection of portions of the complexed or modified sample including the protein or modification may be enhanced by further exposing the sample to an antibody or other reagent specific to the protein or modification, to thereby provide a larger protein target. In the case of detecting protein binding sites, the protein may be labeled prior to binding with the DNA or RNA. Multiple binding sites for a single protein, multiple proteins, or multiple binding sites for multiple proteins or multiple modifications or a mixture of protein complexes and DNA or RNA modifications may all be detected on a single sample.

The nanodetector may be or include a nanopore, or alternatively, it may be or include a fluidic nanochannel or microchannel. In the case of a nanopore, one embodiment includes detection and monitoring of electrical current fluctuations across the nanopore, while in the case of a fluidic channel, an embodiment includes detection and monitoring of an electrical property, such as electrical potential fluctuations, across a detection zone defined by at least one pair of detector electrodes laterally offset along a length of the channel.

Various assay preparation methods are provided herein. In one embodiment, prior to translocation, an additional protein, which may differ from the first protein, may be crosslinked or otherwise bound to the DNA or RNA in a region proximal to the initial protein/DNA or RNA complex or modified DNA/RNA. Additional antibodies or tags that bind to the protein and/or antibodies may be employed to enhance detection. Additionally, prior to translocation, all or a portion of the complex may be coated with a binding moiety to enhance detection. Exemplary binding moieties include proteins such as RecA.

In still another embodiment, a reference genome location map may be superimposed on the DNA or RNA/protein complex or modified DNA/RNA. Such a step simplifies the process by which regions where proteins of interest have bound or modifications are present may be identified, while providing higher resolution location measurements. In this case, detectible probes distinguishable from the complexed portion of the sample, may be hybridized or otherwise bound to or reacted with the DNA or RNA/protein complex or modified DNA/RNA prior to the translocation step. Exemplary probes include oligonucleotide probes, locked nucleic acid (LNA) probes, and peptide nucleic acid (PNA) probes, specific for particular regions of the genome. Alternatively, markers, such as proteins with known specificity or catalytic activities may be used to identify regions of interest relative to a reference genome.

In still another embodiment, when analyzing double-stranded DNA (dsDNA), a nicking enzyme may be used to identify regions of interest relative to a reference genome using, for example, as discussed below, the methods of Patent Application Publication US 2012/0074925 A1, incorporated herein by reference in its entirety. Likewise, in the case of dsDNA, specific DNA binding entities, including major or minor groove binding entities having specificity may be used. In the cases of nicking, specific DNA or RNA binding, and the like, no hybridization to the DNA or RNA is analyzed, but rather another binding or covalent bonding activity. The relative locations of the reference genome marked sites and the protein binding sites or modifications allow the protein binding sites or modifications to be placed more accurately on the reference genome.

Prior to the translocation step, detectible specific DNA or RNA binders may be provided on specific regions of the DNA or RNA/protein complex or modified DNA/RNA.

Upon translocation of the complexed sample through a detection zone, data indicative of the presence of a portion of the DNA or RNA/protein complex or modified DNA/RNA lacking a bound protein or modification, and data indicative of the presence of a portion of the DNA or RNA/protein complex or modified DNA/RNA including a bound protein or modification, is obtained. This data may be assembled to provide a map of binding sites of the protein or modifications on the DNA or RNA sample.

In another aspect, embodiments of the invention include a method for determining sites of protein binding to DNA or RNA or modification sites using a nanodetector. A DNA or RNA/protein complex or modified DNA/RNA to be analyzed is provided and introduced into a nanodetector having a first fluid chamber, a second fluid chamber, a membrane positioned between the first and second chambers and a nanopore extending through the membrane such that the first and second chambers are in fluid communication via the nanopore. The DNA or RNA/protein complex or modified DNA/RNA is introduced into the first chamber and translocated into the second chamber through the nanodetector. During translocation, electrical properties across the nanodetector are detected and monitored, and changes in the electrical property recorded as a function of time. Changes in the electrical property are analyzed to determine at least one site of the DNA or RNA to which a protein is bound or at which a position is modified. Changes in the electrical property allow discrimination of i) the absence of the DNA or RNA/protein complex or modified DNA/RNA in the nanodetector, ii) the presence of a portion of the DNA or RNA/protein complex lacking a bound protein or modified DNA/RNA in the nanodetector, and iii) the presence of a portion of the DNA or RNA/protein complex including a bound protein or modified DNA/RNA in the nanodetector. This data may be employed to provide a map of binding sites of the protein or DNA/RNA modification on the DNA or RNA sample.

In yet another aspect, embodiments of the invention include a method for determining sites of protein binding to DNA or RNA or modifications on the DNA/RNA using a nanodetector employing a fluidic channel such as a nanochannel or microchannel detector. In this embodiment, a DNA or RNA/protein complex or modified DNA/RNA to be analyzed is introduced into a fluidic nanochannel or microchannel having at least one detection volume defined in the fluidic channel by at least one pair of electrodes laterally offset along a length of the channel. The DNA or RNA/protein complex or modified DNA/RNA is translocated through the detection volume, and during translocation, an electrical property in the detection volume is detected. Changes in the electrical property as a function of time are recorded. The changes in the electrical property are analyzed to determine at least one site of the DNA or RNA to which a protein is bound or at which a modification is present.

By recording changes in the electrical property, such as electrical potential measured across the detection volume a function of time, it is possible to discriminate i) the absence of the DNA or RNA/protein complex or modified DNA/RNA in the detection volume, ii) the presence of a portion of the DNA or RNA/protein complex lacking a bound protein or modified DNA/RNA in the detection volume, and iii) the presence of a portion of the DNA or RNA/protein complex including a bound protein or modified DNA/RNA in the detection volume. This data may be employed to provide a map of binding sites of the protein or modifications on the DNA or RNA sample.

Translocation may be achieved, at least in part, by an electrophoretic force provided by electromotive electrodes disposed in the fluidic channel. A pressure gradient, a chemical gradient, or both may be employed as well.

### BRIEF DESCRIPTION OF THE FIGURES

FIG. 1A is a schematic depiction of an assay method in accordance with an embodiment of the invention showing a DNA molecule having a bound protein in a nanodetector apparatus.

FIG. 1B is a schematic depiction of an assay method in accordance with an embodiment of the invention showing a current measurement waveform as a DNA molecule having a bound protein translocates through the nanodetector apparatus of FIG. 1A.

FIG. 2 is a schematic depiction of an assay method in accordance with an embodiment of the invention showing a fluidic nanochannel or microchannel apparatus useful for conducting assays.

FIG. 3A is a schematic depiction of an assay method in accordance with an embodiment of the invention showing an electrical potential measurement as a DNA molecule having a bound protein enters a detection volume in the apparatus of FIG. 2.

FIG. 3B is a schematic depiction of an assay method in accordance with an embodiment of the invention showing an electrical potential measurement as a bound protein on a DNA molecule enters a detection volume in the apparatus of FIG. 2.

FIG. 3C is a schematic depiction of an assay method in accordance with an embodiment of the invention showing an electrical potential measurement as a bound protein on a DNA molecule exits a detection volume in the apparatus of FIG. 2.

FIG. 3D is a schematic depiction of an assay method in accordance with an embodiment of the invention showing an electrical potential measurement as a DNA molecule having a bound protein exits a detection volume in the apparatus of FIG. 2.

FIG. 4 is a schematic depiction of an assay method in accordance with an embodiment of the invention showing a fluidic nanochannel or microchannel apparatus having multiple detection volumes.

### DETAILED DESCRIPTION

A protocol and system for determining sites at which proteins directly bind to DNA or RNA, modify other proteins including histones, or bind to other proteins and sites at which DNA or RNA is modified is described herein. Rather than requiring the rigorous, labor-intensive methodology required by the ChIP-Seq or Mod-Seq methods, embodiments of the present invention offer a simplified, highly accurate means for studying protein interactions with DNA or RNA or sites of DNA or RNA modification. The protocol may offer at least some of the same advantages of ChIP-Seq (reviewed in Park, P J (2009) Nature Rev Genet. 10: 669-680, ChIP-seq: Advantages and challenges of a maturing technology, incorporated herein by reference in its entirety) as well as provide additional benefits by providing long-range interactions and the possibility of mapping multiple proteins or marks or DNA/RNA modifications simultaneously.

As described above, in standard ChIP-Seq experiments, proteins are crosslinked to DNA within the cell or biological system using formaldehyde or similar chemical agents. Under the ChIP-Seq methodology, the DNA is then fragmented. Embodiments of the present invention eliminate this step and maintain intact DNA, thereby allowing longer range information to be generated. In particular, after a DNA/protein complex having crosslinked proteins or proteins otherwise bound to the DNA or DNA/RNA modifications is provided, antibodies or other proteins/reagents that specifically bind the protein or modification of interest are allowed to interact with the DNA/protein complex or modified DNA/RNA. Depending on how strong that interaction is and how crosslinked the DNA/protein complex is, this material can either be run directly in a nanodetector system of the type described below, or processed further for improved performance. For example, the protein may be provided with an appropriate tag, either prior or subsequent to binding with the DNA or modified DNA using any of a variety of methods known in the art.

In yet another embodiment of the invention, after the initial DNA/protein complex or modified DNA/RNA is provided, other entities detectible by the nanodetector system may be employed in place of the antibodies or other proteins/reagents that specifically bind the protein or modification of interest. Thus, dendrimers or silver or gold particles which may be bound to the protein or modification may be used to enhance detection of the protein or modified DNA/RNA in the nanodetector system. It should be understood that this embodiment is not intended to be limited to the use of dendrimers or gold or silver particles, but rather, it is intended that any known entity that may be bound to the protein or modified DNA/RNA to enhance detection is contemplated herein.

In yet another embodiment, examination of multiple proteins or DNA/RNA modifications in the same sample can provide useful information. With current ChIP seq protocols, multiple immunoprecipitations or co-immunoprecipitations are carried out with the resulting information subsequently assembled for a full story (see for example, the multiple assays carried out in Anderson et al (2012) J. Clinical Investigation 122: 1907-1919, “Nkx3.1 and Myc crossregulate shared target genes in mouse and human prostate tumorigenesis” incorporated herein by reference in its entirety). By examining the binding or modified sites directly rather than indirectly by immunoprecipitation, a more direct picture of protein binding and DNA/RNA modifications can be obtained using the inventive protocol.

In yet another embodiment, it may be advantageous to superimpose a reference genome location map on the DNA/protein complex and DNA/RNA modifications. Embodiments of the present invention allow the researcher to mark particular sequences on the reference genome, thereby simplifying the process by which one may identify regions where proteins of interest have bound and DNA/RNA modifications are located while providing higher resolution location measurements. In this case, probes including oligonucleotide probes, locked nucleic acid (LNA) probes, and peptide nucleic acid (PNA) probes, specific for known regions of the genome, may be hybridized or otherwise bound to or reacted with the genomic DNA and processed as described herein. Such sequence specific probes may be constructed such that they are distinguishable from the complexed or modified portion of the sample; however, if the sample was previously mapped such markers need not be distinguishable. Alternatively, markers, such as proteins with known specificity may be used to identify regions of interest relative to a reference genome. In still another embodiment, when analyzing double-stranded DNA (dsDNA), a nicking enzyme may be used to identify regions of interest relative to a reference genome using, for example, the methods of previously mentioned Patent Application Publication US 2012/0074925 A1. Likewise, in the case of dsDNA, specific DNA binding entities, including major or minor groove binding entities having specificity may be used. In the cases of nicking, specific DNA binding, and the like, no hybridization to the DNA or RNA is analyzed, but rather another binding or covalent bonding activity. The relative locations of the reference genome marked sites and the protein binding sites allow the protein binding sites and DNA/RNA modifications to be placed more accurately on the reference genome.

In particular, a nicking enzyme may be utilized by performing the steps of a) providing a double-stranded DNA template having first and second DNA strands, each strand having a 5′ end and a 3′ end, b) contacting the double-stranded DNA template with a nicking endonuclease to form a nick at a sequence-specific nicking location on the first DNA strand, and c) conducting a base extension reaction on the first DNA strand along the corresponding region of the second DNA strand, the reaction starting at the nick and progressing toward the 3′ end of the first DNA strand to thereby form a single-stranded flap on the template adjacent to the nicking location. Optionally, an additional step may be carried out as follows: d) coating the double-stranded DNA template with a binding moiety that enhances electrical detection of the template and the single-stranded flap.

Nicking endonucleases useful in embodiments of the present invention include Nb.BbvCI, Nb.BsmI, NbBsrDI, Nb.BtsI, Nt.AlwI, Nt.BbvCI, Nt.BsmAI, Nt.BspQI, Nt.BstNBI, and Nt.CviPII, used either alone or in various combinations. As noted above, nickases are sequence-specific endonucleases which are characterized in that they cleave only one strand of double-stranded DNA at the recognition site.

The nickase Nb.BbvCI is derived from an E. coli strain expressing an altered form of the BbvCI restriction genes [Ra+:Rb(E177G)] from Bacillus brevis. It nicks at the following recognition site (with “\check{}” specifying the nicking site and “N” representing any one of C, A, G or T):

5′ ...C C T C A G C... 3′
3′ ...G G A G T\check{ }C G... 5′

The nickase Nb.BsmI is derived from an E. coli strain that carries the cloned BsmI gene from Bacillus stearothermophilus NUB 36. It nicks at the following recognition site:

5′ ...G A A T G C N... 3′
3′ ...C T T A C\check{ }G N... 5′

The nickase Nb.BsrDI is derived from an E. coli strain expressing only the large subunit of the BsrDI restriction gene from Bacillus stearothermophilus D70. It nicks at the following recognition site:

5′ ...G C A A T G N N... 3′
3′ ...C G T T A C\check{ }N N... 5′

The nickase Nb.BtsI is derived from an E. coli strain expressing only the large subunit of the BtsI restriction gene from Bacillus thermoglucosidasius. It nicks at the following recognition site:

5′ ...G C A G T G N N... 3′
3′ ...C G T C A C\check{ }N N... 5′

The nickase Nt.AlwI is an engineered derivative of AlwI which catalyzes a single-strand break four bases beyond the 3′ end of the recognition sequence on the top strand. It is derived from an E. coli strain containing a chimeric gene encoding the DNA recognition domain of AlwI and the cleavage/dimerization domain of Nt.BstNBI. It nicks at the following recognition site:

5′ ...G G A T C N N N N\check{ }N... 3′
3′ ...C C T A G N N N N N... 5′

The nickase Nt.BbvCI is derived from an E. coli strain expressing an altered form of the BbvCI restriction genes [Ra(K169E):Rb+] from Bacillus brevis. It nicks at the following recognition site:

5′ ...C C\check{ }T C A G C... 3′
3′ ...G G A G T C G... 5′

The nickase Nt.BsmAI is derived from an E. coli strain expressing an altered form of the BsmAI restriction genes from Bacillus stearothermophilus A664. It nicks at the following recognition site:

5′ ...G T C T C N\check{ }N... 3′
3′ ...C A G A G N N... 5′

The nickase Nt.BspQI is derived from an E. coli strain expressing an engineered BspQI variant from BspQI restriction enzyme. It nicks at the following recognition site:

5′ ...G C T C T T C N\check{ }... 3′
3′ ...C G A G A A G N ... 5′

The nickase Nt.BstNBI catalyzes a single strand break four bases beyond the 3′ side of the recognition sequence. It is derived from an E. coli strain that carries the cloned Nt.BstNBI gene from Bacillus stereothermophilus 33M. It nicks at the following recognition site:

5′ ...G A G T C N N N N\check{ }N... 3′
3′ ...C T C A G N N N N N... 5′

The nickase Nt.CviPII cleaves one strand of DNA of a double-stranded DNA substrate. The final product on pUC19 (a plasmid cloning vector) is an array of bands from 25 to 200 base pairs. CCT is cut less efficiently than CCG and CCA, and some of the CCT sites remain uncleaved. It is derived from an E. coli strain that expresses a fusion of Mxe GyrA intein, chitin-binding domain and a truncated form of the Nt.CviPII nicking endonuclease gene from Chlorella virus NYs-1. It nicks at the following recognition site:

5′ ...\check{ }C C D... 3′
3′ ... G G H... 5′

Each of the restriction endonucleases described above is available from New England Biolabs of Ipswich, Mass.

It should be understood that the invention is not intended to be limited to the nicking endonucleases described above; rather, it is anticipated that any endonuclease capable of providing a nick in a double-stranded DNA molecule may be used in accordance with the methods of the present invention.

Nanodetectors offer a valuable means of determining sites of protein binding and modification. In embodiments of the present invention, instead of requiring an immunoprecipitation step, the sites of antibody binding can be determined directly by measuring changes in electrical properties in a detector as the DNA/protein complex or modified DNA/RNA translocates through the detector.

In a broad embodiment of the invention, one assay preparation methodology includes the following steps.

As a first step, a DNA/protein complex or DNA with modified nucleotides is provided. This analyte may be obtained by isolating DNA fragments having bound proteins or modified nucleotides from a biological sample, such as a cell or cell lysate, or by creating such complexes in vitro. In the latter instance, the DNA fragment or fragments to be studied may be isolated and then contacted with the protein or proteins of interest in the laboratory. Proteins, modified proteins, or other molecules of interest may be crosslinked or otherwise bound to DNA using any of a wide variety of methods known in the art, including exposure to formaldehyde or UV light, to provide the DNA/protein complex or modified DNA. DNA nucleotides may be marked using chemical labels or other tags.

This complex or modified DNA may then be treated with an antibody or other reagent that is specific to the protein or DNA modification of interest.

Note that although embodiments of the invention are intended to apply to proteins, modified proteins, RNAs and other molecules of interest, the description of the assay preparation protocol herein will refer only to proteins. This terminology is intended for purposes of simplification only, and is not intended to limit the scope of the claimed invention.

Once the DNA/protein complex or DNA modification has been treated with an antibody or other reagent that is specific to the protein/modification of interest, it may then be translocated through a nanodetector of the type described below. Changes in an electrical property of the nanodetector may be recorded over time, and analyzed to create a map of protein binding sites or modifications.

Various modifications of the assay preparation method may be employed, alone or in various combinations, to offer further performance enhancements. For example, as noted above, in cases where the protein is sufficiently large, the use of an antibody, or other protein binding reagent may be omitted. Alternatively, other detectable entities, including, but not limited to, gold or silver particles may be bound to the protein or DNA modification to enhance its ability to be detected.

In other embodiments, an antibody or other reagent may be crosslinked or otherwise bound to DNA that is proximal to the protein binding site, or as noted above, specific regions of the DNA/protein complex or DNA modification may be provided with known sequence-specific probes to provide improved localization of protein binding and/or modification sites.

In a further embodiment of the invention, the DNA/protein complex may be coated to enhance its ability to be detected by increasing the signal-to-noise ratio in nanopore or fluidic channel translocation of biomolecules. A DNA or RNA/protein complex or modified DNA or RNA may be incubated with a protein or enzyme that binds to the biomolecule and forms at least a partial coating along the biomolecule. Coating methods are described in detail in co-pending US Patent Application Publication No. 20100243449, incorporated herein by reference in its entirety, and are discussed below.

Broadly, coated biomolecules typically have greater uniformity in their translocation rates, which leads to a decrease in positional error and thus more accurate detection. Due to its increased viscous drag, a coated biomolecule generally translocates through a sequencing system at a slower speed than a non-coated biomolecule. The translocation is preferably slow enough so that a signal can be detected during its passage from a first chamber into a second chamber.

Exemplary binding moieties include proteins such as, for example, RecA, T4 gene 32 protein, f1 geneV protein, human replication protein A, Pf3 single-stranded binding protein, adenovirus DNA binding protein, and E. coli single-stranded binding protein. In particular, RecA protein from E. coli typically binds single- or double-stranded DNA in a cooperative fashion to form filaments containing the DNA in a core and an external sheath of protein (McEntee, K.; Weinstock, G. M.; Lehman, I. R. Binding of the RecA Protein of Escherichia coli to Single- and Double-Stranded DNA. J. Biol. Chem. 1981, 256, 8835). DNA has a diameter of about 2 nm, while DNA coated with RecA has a diameter of about 10 nm. The persistence length of the DNA increases to around 950 nm, in contrast to 0.75 nm for single-stranded DNA or 50 nm for double-stranded DNA. T4 gene 32 protein is known to cooperatively bind single-stranded DNA (Alberts, B. M.; Frey, L. T4 Bacteriophage Gene 32: A Structural Protein in the Replication and Recombination of DNA. Nature, 1970, 227, 1313-1318). E. coli single-stranded binding protein binds single-stranded DNA in several forms depending on salt and magnesium concentrations (Lohman, T. M.; Ferrari, M. E. Escherichia Coli Single-Stranded DNA-Binding Protein Multiple DNA-Binding Modes and Cooperativities. Ann. Rev. Biochem. 1994, 63, 527-570). The E. coli single-stranded binding protein may form a varied coating on the biomolecule. The f1 geneV protein is known to coat single-stranded DNA (Terwilliger, T. C. Gene V Protein Dimerization and Cooperativity of Binding of poly(dA). Biochemistry 1996, 35, 16652), as is human replication protein A (Kim, C.; Snyder, R. O.; Wold, M. S. Binding properties of replication protein A from human and yeast cells. Mol. Cell. Biol. 1992, 12, 3050), Pf3 single-stranded binding protein (Powell, M. D.; Gray, D. M. Characterization of the Pf3 single-strand DNA binding protein by circular dichroism spectroscopy. Biochemistry 1993, 32, 12538), and adenovirus DNA binding protein (Tucker, P. A.; Tsernoglou, D.; Tucker, A. D.; Coenjaerts, F. E. J.; Leenders, H.; Vliet, P. C. Crystal structure of the adenovirus DNA binding protein reveals a hook-on model for cooperative DNA binding. EMBO J. 1994, 13, 2994). Translocation of protein-coated DNA through a nanopore has been demonstrated with RecA bound to double-stranded DNA (Smeets, R. M. M.; Kowalczyk, S. W.; Hall, A. R.; Dekker, N. H.; Dekker, C. Translocation of RecA-Coated Double-Stranded DNA through Solid-State Nanopores. Nano Lett. 2009). The protein coating functions in the same manner for single-stranded DNA and double-stranded DNA.

It should be understood that while the methods of the present invention are not intended to be limited to specific analyses, various known protein assays lend themselves well to the methods described herein. In one non-limiting example, DNA adenine methyltransferase identification, (DamID), (van Steensel, B, et al., (April 2000), Nat. Biotechnol, 18(4): 424-8), incorporated herein by reference in its entirety, is a protocol used to map binding sites of DNA- and chromatin-binding proteins in eukaryotes. In DamID, a fusion protein is formed from DNA adenine methyltransferase, (Dam), and a DNA-binding protein of interest. The DNA-bound fusion protein localizes the methyltransferase in the region of the binding site. This results in the methylation of adenines in GATC sequences close to the protein binding sites. Because adenosine methylation does not occur naturally in eukaryotes, detection of adenine methylation on the target analyte suggests that the fusion protein is or was bound to that target and further suggests that the binding site was at a nearby location. Thus, the methylation sites serve as permanent markers that can be detected using the methods of the present invention. It is anticipated that one may wish to determine the protein binding sites over a course of time. Thus one could identify all methylation sites on the target analyte, thereby determining various location where the protein has bound over time.

In another non-limiting example, the methods of the present invention may be used in connection with proximity utilizing biotinylation and native chromatin immunoprecipitation, (PUB-NChIP) (Shoaib, M., et al., Genome Res., 2013 February; 23(2):331-40), incorporated herein by reference in its entirety. In that protocol, a protein of interest, such as a transcription factor or other nuclear protein, is fused to the bacterial biotin BirA. This fusion protein is coexpressed with the fusion product of a histone and a biotin acceptor peptide (BAP) which is specifically biotinylated by BirA. Upon incorporation of the BAP/histone into chromatin, chromatin regions located in proximity to the BirA/protein complex of interest become preferentially biotinylated. Following the application of streptavidin or other biotin-binding protein, these sites are then detectable using the methods of the present invention. This method is particularly useful for proteins that bind to histones, but not DNA, since, while they would not be detectable in a crosslinking study, the protocol may leave permanent detectable markers on a histone or other protein.

Although the two methods given in detail above are simply examples, it should be understood that the methods of the present invention are intended to provide a broad ability to detect protein and other modifications on DNA and RNA whether they occur naturally or not. As such, the methods of the present invention are intended to include the detection of modifications including, but not limited to, methylations, hydroxylations, and glucosylations.

The translocation rate or frequency through the nanodetector may be further regulated by introducing either one or both of a pressure gradient or a chemical (salt) gradient between the chambers. Exemplary salt concentration ratios of the cis to the trans side of the chamber may include, but are not limited to, 1:2, 1:4, 1:6, and 1:8. For example, salt concentrations may range from about 0.5 M KCl to about 1 M KCl on the cis side and from about 1 M KCl to about 4 M KCl on the trans side. The signal is preferably strong enough to be detected using known methods or methods described herein. Exemplary signal-to-noise ratios include, but are not limited to, 2:1, 5:1, 10:1, 15:1, 20:1, 50:1, 100:1, and 200:1. With a higher signal-to-noise ratio, a lower voltage may be used to effect translocation.

The translocation rate and frequency may also be further regulated by applying pressure to either the cis or trans side of the fluidic cell.

The analytes described herein may be configured for detection of positional information in a nanodetector using a nanopore and/or a fluidic channel, i.e., a fluidic microchannel or nanochannel system. Mapping of analytes may be carried out using electrical detection methods employing nanopores, nanochannels, or microchannels using the methods described in U.S. Patent Publication No. 2010-0310421, incorporated herein by reference in its entirety.

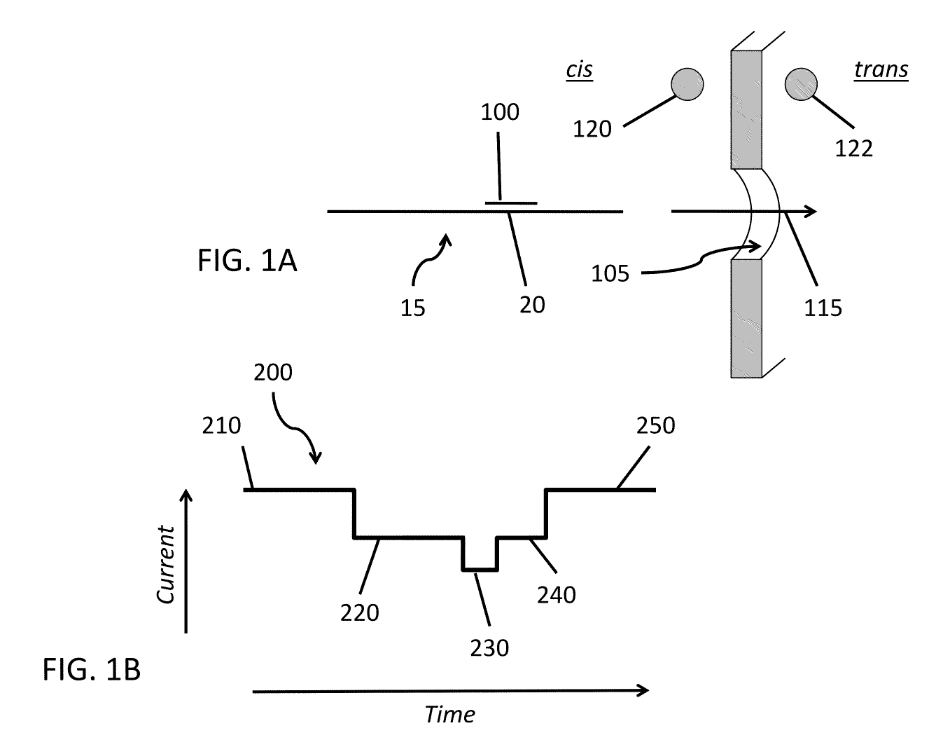

In one embodiment, current across a nanopore is measured during translocation of a DNA complex through the nanodetector as shown in FIG. 1A. The nanopore may have a diameter selected from a range of about 1 nm to about 1 μm. More preferably the nanopore has a diameter that is between about 2.3 nm and about 100 nm. Even more preferably the nanopore has a diameter that is between about 2.3 nm and about 50 nm. Changes in an electrical property across a nanopore may be monitored as the DNA complex is translocated therethrough, with changes in the electrical property being used to distinguish regions of the analyte including bound proteins, and regions of the analyte lacking such proteins.

Specifically, for nanopore 105, a measurable current 115 produced by electrodes 120, 122 runs parallel to the movement of the target analyte 15, i.e., a DNA fragment 20 having a bound protein 100. The protein may or may not also include an antibody or other protein/reagent (not shown) that interacts with the protein 100 or DNA fragment 20 and enhances its ability to be detected. Likewise, at least a portion of the target analyte 15 may include a coating, such as a RecA coating, to enhance its ability to be detected.

Variations in current are a result of the relative diameter of the target analyte 15 as it passes through the nanopore 105. This relative increase in volume of the target analyte 15 passing through the nanopore 105 causes a temporary interruption or decrease in the current flow through the nanopore, resulting in a measurable current variation. Portions of the target analyte 15 including a bound protein 100, and optional antibody, are larger in diameter than portions of the target analyte that do not include the protein. As a result, when a portion of the target analyte having bound protein 100 passes through the nanopore 105, further interruptions or decreases in the current flow between electrodes 120, 122 occurs. These changes in current flow are depicted in the waveform 200 in FIG. 1B.

Analysis of the waveform 200 permits differentiation between regions of the analyte including proteins and regions without proteins, based, at least in part, on the detected changes in the electrical property, to thereby determine protein binding locations on at least a portion of the DNA template. In FIG. 1B, the waveform 200 depicts the changes in a detected electrical property as the analyte 15 passes through the nanodetector, and may be interpreted as follows. Current measurement 210 represents measured background current, i.e., baseline current, prior to passage of the DNA molecule 20 and protein 100, through the nanopore 105 from the cis side to the trans side. As the analyte 15 enters the nanopore 105, from the cis side of the nanopore, the current is partially interrupted forming a first trough 220 in the recorded current. Once the portion of the analyte having bound protein 100 and optional antibody enters the nanopore 105, a further decrease in current occurs, causing a deeper, second trough 230 in the current measurement. Upon passage of the protein and antibody 100 entirely through the nanopore 105, a distal portion of the DNA template 20 may remain in the nanopore. This causes the measured current 240 to rise to approximately the level of the first trough 220. Finally, once the entire analyte 15 has passed completely through the nanopore 105 to the trans side, the measured current 250 returns to a background baseline level approximating that of the initial level 210. The current variation measurements are recorded as a function of time.

As a result, the periodic variations in current indicate where, as a function of relative or absolute position, proteins 100 have bound to regions on the DNA template 20. Since the proteins are bound at specific sites, the relative or absolute position of the sites associated with protein binding for the specific protein or DNA modification employed may be determined. This allows mapping of those specific protein binding and DNA modification sites on the analyte. Multiple maps produced using multiple proteins or DNA modifications may be generated.

As noted above, the use of a binding moiety or coating, such as the protein RecA, may further enhance detection of analytes and complexed protein regions on analytes because the added bulk of the binding moiety coating causes greater current deflections.

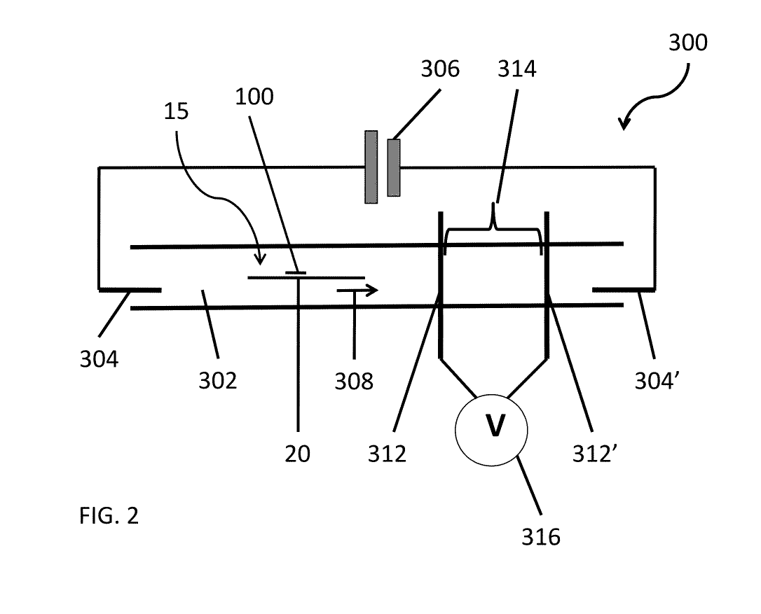

In another embodiment, an electrical property such as electrical potential or current is measured during translocation of a protein/DNA complex through a nanodetector comprising a nanochannel or microchannel as shown in FIGS. 2 through 4. One embodiment of a fluidic channel apparatus is shown schematically in FIG. 2. In FIG. 2, the apparatus 300 includes a fluidic microchannel or nanochannel 302. The fluidic channel may be a microchannel having a width selected from a range of about 1 μm to about 25 μm or a nanochannel having a width selected from a range of about 10 nm to about 1000 nm. In the case of a microchannel, the depth may be selected from a range of about 200 nm to about 5 μm, whereas in the case of a nanochannel, the depth may be selected from a range of about 10 nm to about 1000 nm. In either case, the channel may have a length selected from a range of about 200 nm to about 10 cm. The nanochannel or microchannel may be formed by any number of nano- and micro-fabrication methods known in the art. In one embodiment, the channel may be fabricated as a trench in a substrate which is subsequently capped.

A first pair of electromotive electrodes 304, 304′ is connected to a voltage source 306 and positioned in a manner to provide an electrical potential along at least a portion of the length of the channel. Thus, when a potential is applied to the electromotive electrodes, these electrodes provide an electrical current along the channel and may be used to provide or enhance a driving force 308 to an analyte 15 in the channel. As before, the analyte 15 includes a DNA template 20 having one or more bound proteins or DNA modifications 100. Also as before, the protein may or may not include an antibody or other protein/reagent (not shown) that interacts with the protein or DNA modification 100 or DNA fragment 20 and enhances its ability to be detected. Other driving forces such as pressure or chemical gradients are contemplated as well. A second pair of electrodes 312, 312′, i.e., detector electrodes, is positioned preferably substantially perpendicular to the channel in a spaced apart, i.e., laterally offset, relationship to define a detection volume 314. The second pair of detector electrodes 312, 312′ is connected to a detector 316, such as a voltmeter, which monitors an electrical property in the detection volume 314. In an embodiment where the detector 316 is a voltmeter, an electrical potential between the pair of detector electrodes 312, 312′, is measured across the detection volume 314.

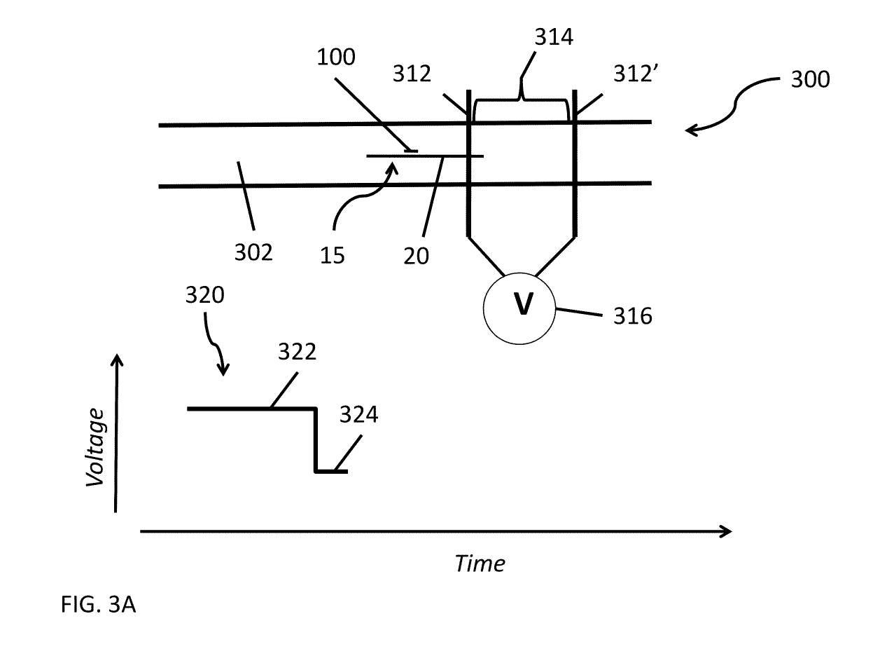

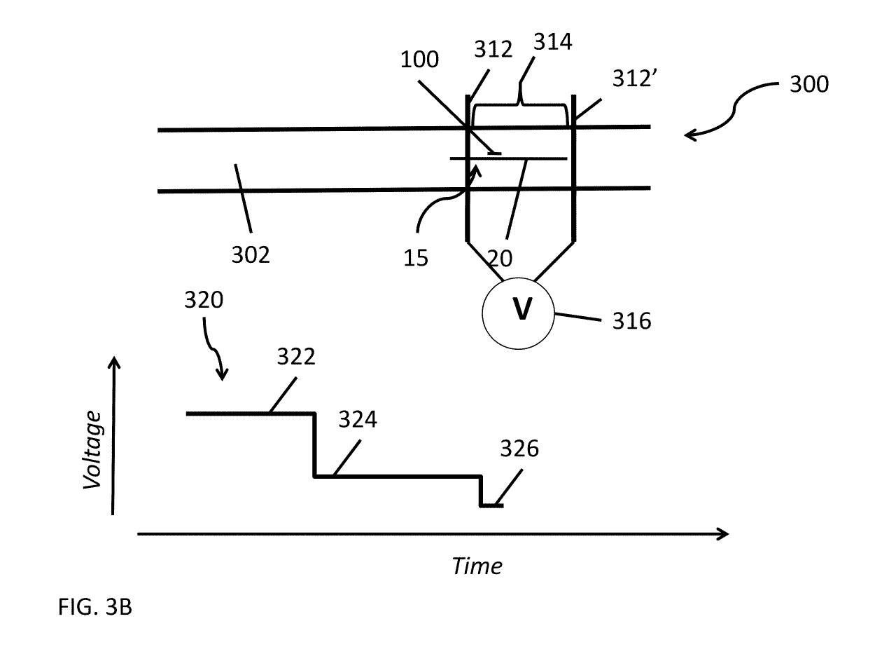

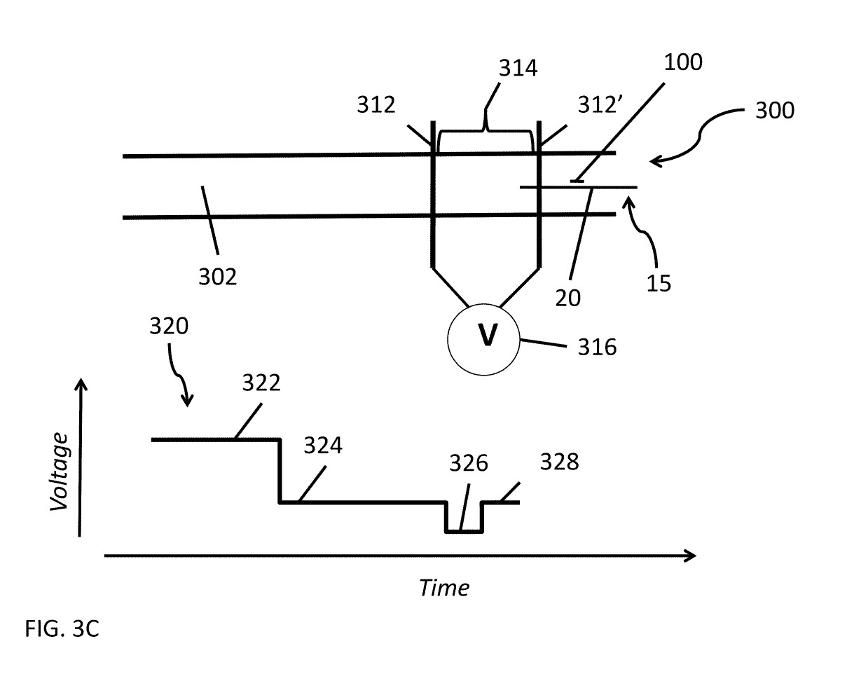

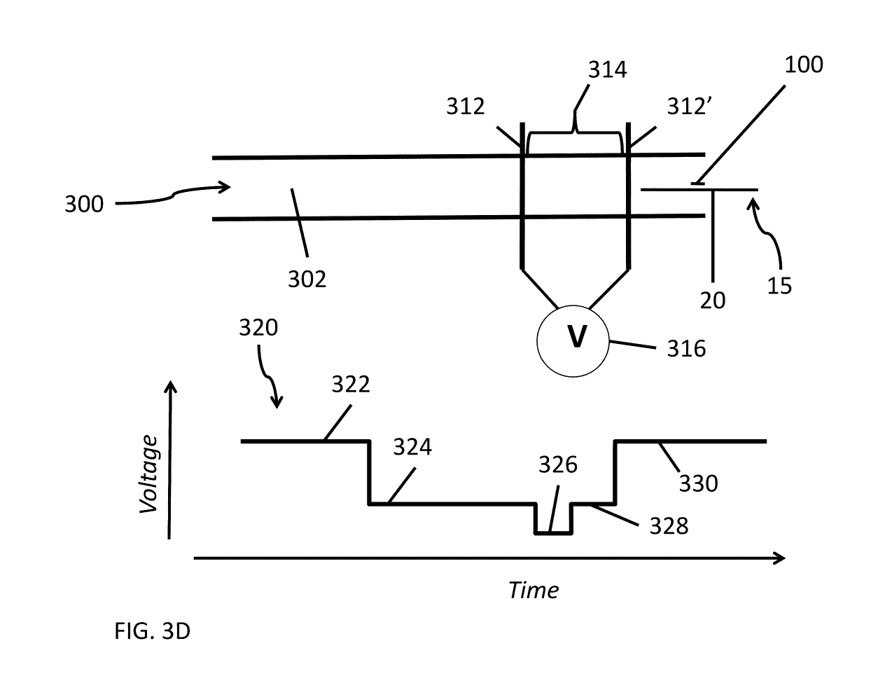

The operation of the device is depicted schematically in FIGS. 3A-3D in which changes in an electrical property across a fluidic channel are monitored, as the analyte 15 is translocated therethrough. The changes in the electrical property are indicative of the presence or absence of the analyte as well as protein-bound and protein-free regions. In FIGS. 3A-3D, the electromotive electrodes 304, 304′ and the current source 306 have been omitted for clarity. In FIG. 3A, the fluidic channel 302 contains an analyte 15, which translocates therethrough. An electrical property, in this case electrical potential, is measured and recorded across the detection volume 314 by the detector electrodes 312, 312′ and the detector 316. The analyte 15 is a DNA template 20 upon which proteins or DNA modifications 100 and optionally antibodies or other entities have been bound using the methods described previously. The DNA template and/or the protein and antibody may be coated with a binding moiety, such as the protein RecA, to enhance detection.

Prior to the entry of the analyte 15 into the detection volume 314, a substantially constant baseline background voltage 322 is measured across the detection volume. This voltage is shown in the waveform 320 of FIG. 3A. It should be noted that while this example monitors changes in voltage across the detection volume, voltage is used merely for simplicity of description. Embodiments of the invention are not intended to be limited solely to voltage measurements; rather, any of a wide variety of electrical properties may be monitored and analyzed. As the analyte 15 enters the detection volume 314, it may cause an interruption or decrease in the electrical property measured in the detection volume. This interruption or decrease causes a first trough 324 to be exhibited in the waveform 320.

FIG. 3B shows the device and waveform 320 once a portion of the target analyte 15 including the protein 100 has entered the detection volume 314. Entry of the protein into the detection volume 314 causes a further interruption or decrease in the electrical property measured in the detection volume. This further interruption or decrease causes a second trough 326 to be exhibited in the waveform 320.

In FIG. 3C, the portion of the analyte 15 containing the protein 100 has exited the detection volume 314; however, a distal portion of the analyte 15 is still present in the detection volume. As a result, the waveform 320 has returned to a level 328 approximating that detected when the initial portion of the analyte 15 first entered the detection volume.

Finally, as shown in FIG. 3D, the analyte 15 has fully exited the detection volume 314. As a result, the waveform 320 has returned to a background level 330 approximating that detected prior to initial entry of the analyte 15 into the detection volume 314. Analysis of the waveform 320 permits differentiation between protein-containing and protein-free regions of the analyte, based, at least in part, on the detected changes in the electrical property. As such, it is possible to determine protein binding and modified DNA locations and map them on at least a portion of the analyte.

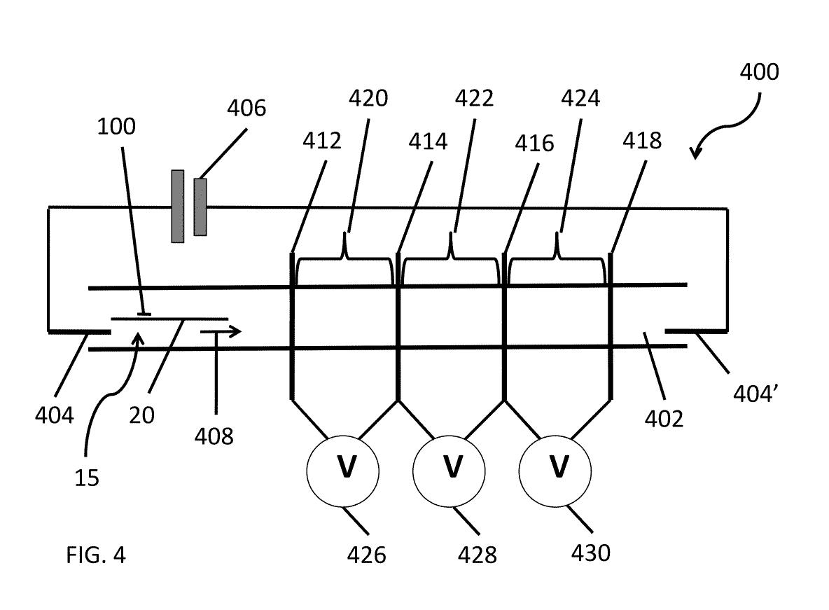

Another embodiment of a fluidic channel apparatus is shown in FIG. 4. In FIG. 4, the apparatus 400 includes a fluidic microchannel or nanochannel 402. As before, the fluidic channel may be a microchannel having a width selected from a range of about 1 μm to about 25 μm or a nanochannel having a width selected from a range of about 10 nm to about 1000 nm. In the case of a microchannel, the depth may be selected from a range of about 200 nm to about 5 μm, whereas in the case of a nanochannel, the depth may be selected from a range of about 10 nm to about 1000 nm. In either case, the channel may have a length selected from a range of about 200 nm to about 10 cm.

A first pair of electromotive electrodes 404, 404′ is connected to a voltage source 406 and positioned in a manner to provide an electrical potential along at least a portion of the length of the channel. When a potential is applied to the electromotive electrodes, these electrodes provide an electrical current along the channel and may be used to provide or enhance a driving force 408 to an analyte 15 in the channel. As before, the analyte 15 includes a DNA template 20 having one or more bound proteins or DNA modifications 100. Also as before, the protein may or may not include an antibody or other protein/reagent (not shown) that interacts with the protein 100 or DNA fragment 20 and enhances its ability to be detected. Other driving forces such as pressure or chemical gradients are contemplated as well.

Multiple detector electrodes 412, 414, 416, 418, are positioned preferably perpendicular to the channel in a laterally offset, spaced apart relationship to define a plurality of detection volumes between adjacent detector electrodes. Thus, as seen in FIG. 4, detector electrodes 412 and 414 define detection volume 420, detector electrodes 414 and 416 define detection volume 422, and detector electrodes 416 and 418 define detection volume 424. The detector electrodes are each connected to detectors 426, 428, 430 such as voltmeters, which monitor an electrical property in each detection volume. In the embodiment where the detectors are voltmeters, a drop in electrical potential is measured across each detection volume. Operation of the apparatus is similar to that of the system of FIGS. 3A-3D, with the exception that additional waveforms are generated due to the presence of additional detection volumes. The additional waveforms may be combined to further improve the quality of the data being generated by the device.

It should be understood that number of detector electrodes and detection volumes is not intended to limited to those depicted in FIG. 4. Rather, any number of detection volumes may be included along the length of the fluidic channel. Further, the detector electrodes and detection volumes need not be evenly spaced, evenly sized or directly adjacent to one another. Various detection volume sizes, spacing and configurations are contemplated.

As noted above previously, the methods and systems described above offer the ability to determine specific sites of DNA modification or at which specific proteins directly bind to DNA, modify other proteins including histones, or bind to other proteins. This provides valuable information for the development of therapeutics and therapeutics targets, evaluation of therapeutic safety and efficacy, and disease diagnosis and prognosis. For example, the protein families involved in directing changes to the epigenome and various therapeutics that are effective in causing changes are reviewed in Arrowsmith et al (2012) Nature Rev Drug Discovery 11: 384-400, “Epigenetic protein families: a new frontier for drug discovery,” incorporated herein by reference in its entirety. Being able to monitor the impact such therapeutics at a whole genome level will be advantageous for improving such drugs and monitoring them both for efficacy and potential side effects. Additionally, the location or frequency of epigenetic marks can be a useful predictor for health and disease (Greer and Shi (2012) Nature Reviews Genetics 13: 343-357, “Histone methylation: a dynamic mark in health, disease and inheritance”), incorporated herein by reference in its entirety. Differential protein binding to particular genes detected by ChIP seq can also be used to predict clinical outcomes in cancer and other diseases (Ross-Innes et al, (2012) Nature 481: 389-393, “Differential oestrogen receptor binding is associated with clinical outcome in breast cancer”), incorporated herein by reference in its entirety. Being able to more quickly and reproducibly detect such changes would enable better treatment decisions. Similarly, the linkage of DNA methylation with a variety of disease states has been described and its increasing importance suggested (Heyn and Esteller, (2012) Nature Rev. Genet. 13: 679-692), “DNA methylation profiling in the clinic: applications and challenges”), incorporated herein by reference in its entirety. Thus, even with current technology, knowing the locations of epigenetic and transcription factor binding sites and DNA modifications provides many benefits. Being able to generate maps for multiple proteins and DNA modifications and over a longer distance range should only enhance those benefits.

### EQUIVALENTS

Those skilled in the art will readily appreciate that all parameters listed herein are meant to be exemplary and actual parameters depend upon the specific application for which the methods and materials of embodiments of the present invention are used. It is, therefore, to be understood that the foregoing embodiments are presented by way of example only and that, within the scope of the appended claims and equivalents thereto, the invention may be practiced otherwise than as specifically described.

The described embodiments of the invention are intended to be merely exemplary and numerous variations and modifications will be apparent to those skilled in the art. All such variations and modifications are intended to be within the scope of the present invention as defined in the appended claims.

What is claimed is:

1. A method for determining sites of protein binding to DNA or RNA or sites of modification of DNA or RNA, the method comprising the steps of:
a) providing a sample comprising a DNA/protein complex, an RNA/protein complex, modified DNA or modified RNA to be analyzed;
b) translocating the sample through a nanodetector having a detection zone;
c) detecting and monitoring an electrical property in the detection zone; and
d) analyzing the electrical property to determine at least one site of the sample to which a protein is bound or at which the sample is modified,
wherein changes in the electrical property allow discrimination of i) the absence of the sample in the detection zone, ii) the presence of a portion of the sample lacking a bound protein or modification in the detection zone, and iii) the presence of a portion of the sample including a bound protein or modification in the detection zone.

2. The method of claim 1, wherein the sample is isolated from a biological sample.

3. The method of claim 1, wherein the sample is created in vitro.

4. The method of claim 1, wherein the protein of the DNA/protein complex or RNA/protein complex is crosslinked to the DNA or RNA.

5. The method of claim 1, wherein the modification comprises methylation, hydroxylation or glucosylation of the DNA or RNA.

6. The method of claim 1, further comprising, prior to the translocating step, exposing the sample to an antibody or other reagent specific to the protein or modification.

7. The method of claim 1, wherein providing the DNA/protein complex or RNA/protein complex comprises labeling the protein prior to complexation.

8. The method of claim 1, wherein the nanodetector comprises a nanopore.

9. The method of claim 8, wherein the electrical property comprises electrical current across the nanopore.

10. The method of claim 1, wherein the nanodetector comprises a fluidic channel.

11. The method of claim 10, wherein the fluidic channel comprises a nanochannel or a microchannel.

12. The method of claim 11, wherein the detection zone is defined in the fluidic channel by at least one pair of detector electrodes laterally offset along a length of the channel.

13. The method of claim 12, wherein the electrical property comprises an electrical potential between at least one pair of detector electrodes.

14. The method of claim 1, further comprising, prior to the translocating step, binding an additional protein to a proximal region of the DNA/protein complex, the RNA/protein complex, the modified DNA or the modified RNA.

15. The method of claim 14, wherein the additional protein is bound by crosslinking.

16. The method of claim 1, which includes, prior to the translocating step, binding a tag to the DNA/protein complex, the RNA/protein complex, the modified DNA or the modified RNA.

17. The method of claim 14, wherein the additional protein differs from the protein in the DNA/protein or RNA/protein complex.

18. The method of claim 1, which includes, prior to the translocating step, providing detectible probes on specific regions of the DNA/protein complex, the RNA/protein complex, the modified DNA or the modified RNA.

19. The method of claim 18, wherein the probes comprise at least one of oligonucleotide probes, locked nucleic acid probes, peptide nucleic acid probes, proteins that are sequence specific, or combinations thereof.

20. The method of claim 1, further comprising, prior to the translocating step, providing detectible specific DNA or RNA binders on specific regions of the DNA/protein complex, the RNA/protein complex, the modified DNA or the modified RNA.

21. The method of claim 1, wherein analyzing the electrical property comprises analyzing data indicative of the presence of a portion of the sample lacking a bound protein or modification in the detection zone, and data indicative of the presence of a portion of the sample including a bound protein or modification in the detection zone, to provide a map of binding sites of the protein or modification sites on the sample.

22. The method of claim 1, further comprising, prior to translocation, coating at least a portion of the DNA/protein complex, the RNA/protein complex, the modified DNA or the modified RNA.

23. The method of claim 22, wherein the coating comprises a protein that binds to at least a portion of the DNA/protein complex, the RNA/protein complex, the modified DNA or the modified RNA.

24. The method of claim 23, wherein the protein comprises RecA.

25. The method of claim 1, further comprising, prior to the translocating step, providing detectible tags at specific regions of the DNA/protein complex, the RNA/protein complex, the modified DNA or the modified RNA, such regions created by nicking enzymes or other DNA or RNA modification enzymes or binding entities.

26. A method for determining sites of protein binding to DNA or RNA or sites of modification of DNA or RNA, the method comprising the steps of:
a) providing a sample comprising a DNA/protein complex, an RNA/protein complex, modified DNA or modified RNA to be analyzed;
b) providing a nanodetector apparatus having a first fluid chamber, a second fluid chamber, a membrane positioned between the first and second chambers and a nanopore extending through the membrane such that the first and second chambers are in fluid communication via the nanopore;
c) introducing the sample into the first chamber;
d) translocating the sample from the first chamber into the second chamber through the nanopore;
e) detecting and monitoring an electrical property across the nanopore;
f) recording the changes in the electrical property as a function of time; and
g) analyzing the changes in the electrical property to determine at least one site of the sample to which a protein is bound or at which the sample is modified,
wherein changes in the electrical property allow discrimination of i) the absence of the sample in the nanopore, ii) the presence of a portion of the sample lacking a bound protein or modification in the nanopore, and iii) the presence of a portion of the sample including a bound protein or modification in the nanopore.

27. The method of claim 26, wherein analyzing the changes in the electrical property produces a map of binding sites of the protein or sites of modification on the sample.

28. A method for determining sites of protein binding to DNA or RNA or sites of modification of DNA or RNA, the method comprising the steps of:
a) providing a sample comprising a DNA/protein complex, an RNA/protein complex, modified DNA or modified RNA to be analyzed;
b) disposing the sample in a nanodetector having a fluidic nanochannel or microchannel, the fluidic nanochannel or microchannel including at least one detection volume defined by at least one pair of electrodes laterally offset along a length of the fluidic nanochannel or microchannel;
c) translocating the sample through the detection volume;
d) detecting an electrical property in the detection volume as the sample translocates therethrough;
e) recording the changes in the electrical property as a function of time,
f) analyzing the changes in the electrical property to determine at least one site on the sample to which a protein is bound or at which the sample is modified,
wherein changes in the electrical property allow discrimination of i) the absence of the sample in the detection zone, ii) the presence of a portion of the sample lacking a bound protein or modification in the detection zone, and iii) the presence of a portion of the sample including a bound protein or modification in the detection zone.

29. The method of claim 28, wherein translocating the sample comprises providing an electrophoretic force by at least first and second electromotive electrodes disposed in the fluidic nanochannel or microchannel.

30. The method of claim 29, wherein translocating the sample further comprises using at least one of a potential gradient, a pressure gradient, a chemical gradient or combinations thereof.

31. The method of claim 28, wherein the electrical property is an electrical potential measured across the detection volume.

32. The method of claim 28, wherein analyzing the changes in the electrical property produces a map of binding sites of the protein or sites of modification on the sample.
